(19) United States
(12) Patent Application Publication
(10) Pub. No.: US 2006/0142255 A1
(43) Pub. Date: Jun. 29, 2006

(54) Hypocholesteremic preparations containing phytostenolesters of conjugated fatty acids, and methods of reducing serum cholesterol levels using the same

(76) Inventor: Bernd Fabry, Korschenbroich (DE)

Correspondence Address: COGNIS CORPORATION;PATENT DEPARTMENT, 300 BROOKSIDE AVENUE, AMBLER, PA, 19002, US

(21) Appl. No.: 11/250,028

(22) Filed: Oct. 13, 2005

Related U.S. Application Data
(63) Continuation of application No. 09/554,386, filed on Jul. 19, 2000, now abandoned.

(30) Foreign Application Priority Data
Nov. 14, 1997 (DE) 197 50 422.1

(51) Int. Cl. A61K 31/56

(52) U.S. Cl. 514/169; 514/560

(57) ABSTRACT

A hypocholesteremic preparation containing at least one phytostenol ester of a conjugated fatty acid having from about 6 to about 24 carbon atoms is disclosed. Methods of reducing serum cholesterol content in a mammal via administration of hypocholesteremic preparations described herein are also disclosed.

### CROSS-REFERENCE TO RELATED APPLICATIONS

This application claims priority under 35 U.S.C. §120, and is a continuation of U.S. patent application Ser. No. 09/554,386, filed on Jul. 19, 2000 as a 35 U.S.C. §371 submission based upon International Application No. PCT/EP98/07057, having an International Filing Date of Nov. 14, 1997, the entire contents of which are herein incorporated by reference.

### BACKGROUND OF THE INVENTION

Hypocholesteremic active agents are understood as meaning preparations which lead to a decrease in the cholesterol content in the serum of warm-blooded animals without an inhibition or lowering of the formation of cholesterol in the blood occurring. Phytostenols, i.e. plant stenols, and their esters with fatty acids have already been proposed for this purpose by Peterson et al. in J. Nutrit. 50, 191 (1953). The Patent Specifications U.S. Pat. No. 3,089,939, U.S. Pat. No. 3,203,862 as well as the German Laid-Open Specification DE-A 2035069 (Procter & Gamble) also point in the same direction. The active agents are customarily added to cooking or food oils and then ingested via the food, the amounts employed, however, as a rule being low and customarily below 0.5% by weight in order to prevent the food oils from becoming cloudy or the stenols from being precipitated on addition of water. For use in the foodstuffs area, in cosmetics, pharmaceutical preparations and in the agrarian sector, storage-stable emulsions of the stenol esters in sugar or polyglycerol esters are proposed in European Patent Application EP-A1 0289636 (Ashai). The incorporation of sitostanol esters to decrease the blood cholesterol content in margarine, butter, mayonnaise, salad dressings and the like is proposed in European Patent Specification EP-B1 0594612 (Raision).

The disadvantage, however, is that the phytostenol esters can customarily be added to the foodstuffs only in small amounts, as otherwise there is the danger that they will impair the taste and/or the consistency of the preparations. For a lasting effect on the cholesterol content in the blood, however, the intake of larger amounts of phytostenol esters would be desirable. Furthermore, the rate at which the substances decrease the content of cholesterol in the serum is worthy of improvement. The object of the invention consequently consisted in remedying these deficiencies.

### BRIEF SUMMARY OF THE INVENTION

The present invention includes hypocholesteremic preparations comprising phytostenol esters of conjugated fatty acids, and methods of reducing serum cholesterol levels in mammals through administration of such preparations.

The invention provides the use of esters of phytostenols with fatty acids having 6 to 24 carbon atoms and at least two conjugated double bonds, optionally together with potentiating agents selected from the group consisting of tocopherols, chitosans, phytostenol sulfates and/or (deoxy)ribonucleic acids for producing hypocholesteremic preparations.

Surprisingly, it has been found that phytostenol esters based on conjugated fatty acids exhibit, with respect to reducing the cholesterol content in the blood, considerably higher activity than comparable phytostenol esters derived from saturated fatty acids, monounsaturated fatty acids or polyunsaturated fatty acids having two or more unconjugated double bonds. By combining the phytostenol esters to be used according to the invention (component a) with potentiating agents (component b) from the group of the chitosans, phytostenol sulfates and/or deoxy- or ribonucleic acids which for their part have little, if any, hypocholesteremic properties, it is possible to accelerate the reduction of the cholesterol content in the serum further. Moreover, encapsulated in gelatin, both the phytostenol esters and the mixtures of active agents can be taken orally without problems.

### DETAILED DESCRIPTION OF THE INVENTION

Phytostenol Esters

Phytostenols (or synonymously phytosterols) are understood as meaning plant steroids which carry a hydroxyl group only on C-3, but otherwise no functional groups. As a rule, the phytostenols have 27 to 30 carbon atoms and a double bond in the 5/6, optionally 7/8, 8/9 or other positions. The unsaturated stenols can be hydrogenated to give the corresponding saturated stanols, which are likewise embraced by the present invention. Esterification of the stenols or stanols with unsaturated fatty acids having conjugated double bonds, preferably conjugated linoleic acid (CLA) or conjugated fish fatty acids, gives the substances forming the component (a). The phytostenol component of the esters can be derived from ergostenols, campestenols, stigmastenols, brassicastenols, preferably sitostenols or sitostanols and in particular β-sitostenols or β-sitostanols. The preparation can be carried out in a manner known per se, for example by direct esterification of the stenols with the fatty acids and subsequent hydrogenation of the esters, by direct esterification of the stanols with the fatty acids or, preferably, by transesterification and, if appropriate, hydrogenation of the stenols or stanols with the corresponding conjuene fatty acid methyl esters. A general preparation process by transesterification of the stenols/stanols with fatty acid lower alkyl esters or triglycerides in the presence of suitable catalysts, such as, for example, sodium ethylate or especially also enzymes is described in EP-A2 0195311 (Yoshikawa). According to the invention, the fatty acid component of the phytostenol esters may also comprise minor amounts (less than 50 mol %) of saturated, monounsaturated or polyunsaturated non-conjugated proportions. Accordingly, for preparing the esters, it is possible to use, instead of pure conjugated linoleic acid, for example a technical-grade mixture having a high proportion of conjugated linoleic acid, commercially available, for example, under the name Selin® CLA (Grünau). In the same manner, for preparing the phytostenol esters, it is also possible to transesterify the corresponding fatty acid methyl esters or triglycerides (for example Selin® CLA-TG) having a high conjuent content.

Tocopherols

Tocopherols which are suitable as potentiating agents for the phytostenol esters are understood as meaning chroman-6-ols (3,4-dihydro-2-H-1benzopyran-6-ols) substituted in the 2-position by 4,8,12-trimethyltridecyl radicals, which obey the formula (II) 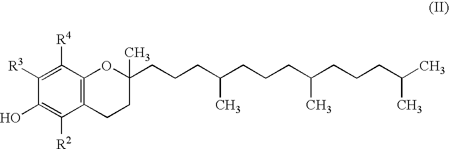
in which R², R³ and R⁴ independently of one another are hydrogen or a methyl group. Tocopherols belong to the bioquinones, i.e. polyprenylated 1,4-benzo- or naphthoquinones whose prenyl chains are saturated to a greater or lesser extent. Typical examples of tocopherols which are possible within the meaning of the invention as component (b1) are ubiquinones, boviquinones, K vitamins and/or menaquinones (2-methyl-1,4-naphthoquinones). In the case of the tocopherols, a differentiation is furthermore made between α, β, γ-, δ- and ε-tocopherols, where the latter can still have the original unsaturated prenyl side chain, and α-tocopherolquinone and -hydroquinone, in which the pyran ring system is opened. Preferably, as component (b), α-tocopherol (vitamin E) of the formula (II) is employed, in which R², R³ and R⁴ are methyl groups, or esters of α-tocopherol with carboxylic acids having 2 to 22 carbon atoms, such as, for example, α-tocopherol acetate or α-tocopherol palmitate.
Chitosans

Chitosans, which are also suitable as potentiating agents (b2) for the phytostenol esters, are biopolymers and are included in the hydrocolloids group. Considered chemically, they are partially deacetylated chitins of different molecular weights, which contain the following—idealized—monomer unit (III)

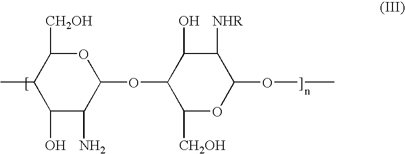

In contrast to most hydrocolloids, which are negatively charged in the biological pH region, chitosans are cationic biopolymers under these conditions. The positively charged chitosans can interact with oppositely charged surfaces and are therefore employed in cosmetic hair- and body-care preparations and pharmaceutical preparations (cf. Ullmann's Encyclopedia of Industrial Chemistry, 5th Ed., Vol. A6, Weinheim, Verlag Chemie, 1986, pp. 231-332). Overviews on this subject have also appeared, for example, by B. Gesslein et al. in HAPPI 27, 57 (1990), O. Skaugrud in Drug Cosm. Ind. 148, 24 (1991) and E. Onsoyen et al. in Seifen-Öle-Fette-Wachse 117, 633 (1991). To produce chitosans, chitin, preferably the shell remains from crustaceans, which are available in large amounts as cheap raw materials, is used as a starting material. In a process which has been described for the first time by Hackmann et al., the chitin is customarily first deproteinated by addition of bases, demineralized by addition of mineral acids and finally deacetylated by addition of strong bases, it being possible for the molecular weights to be distributed over a wide spectrum. Preference is given to using either low-molecular-weight chitosans having an average molecular weight of from about 50,000 to about 250,000 dalton or high-molecular-weight chitosans having an average molecular weight of from about 500,000 to about 2,000,000. Corresponding processes are known, for example, from Makromol. Chem. 177, 3589 (1976) or French Patent Application FR-A 2701266. Particular preference is given to using the types disclosed in the German patent applications DE-A1 4442987 and DE-A1 19537001 (Henkel), which have an average molecular weight of from 800,000 to 1,200,000 dalton, a viscosity according to Brookfield (1% by weight in glycolic acid) below 5000 mpas, a degree of deacetylation in the range from 80 to 88% and an ash content of less than 0.3% by weight. Suitable according to the invention are, in addition to the chitosans as typical cationic biopolymers, also anionic or nonionic derivatized chitosans, such as, for example, carboxylation, succinylation or alkoxylation products, as described, for example, in the German patent DE-C2 3713099 (L'Oréal) and the German patent application DE-A1 19604180 (Henkel).

Phytostenol sulfates

Phytostenol sulfates, which are also suitable as potentiating agents (b3) for the phytostenol esters, are known substances which can be prepared, for example, by sulfation of phytostenols with a complex of sulfur trioxide and pyridine in benzene [cf. J. Am. Chem. Soc. 63, 1259 (1941)]. Typical examples are the sulfates of ergostenols, campestenols, stigmastenols and sitostenols. The phytostenol sulfates can be present as alkali metal and/or alkaline earth metal salts, as ammonium, alkylammonium, alkanolammonium and/or glucammonium salts. As a rule, they are employed in the form of their sodium salts.

(Deoxy)Ribonucleic Acids

(Deoxy)ribonucleic acids (DNA or RNA), which are suitable as the last group of potentiating agents (b4) for the phytostenol esters, are understood as meaning high molecular weight, threadlike polynucleotides which are derived from 2′-deoxy-β-D-ribonucleosides or D-ribonucleosides, which for their part in turn are synthesized from equivalent amounts of a nucleobase and the pentose 2-deoxy-D-ribofuranose or D-ribofuranose. As nucleobases, the DNA or RNA can contain the purine derivatives adenine and guanine and also the pyrimidines cytosine and thymine or uracil. In the nucleic acids, the nucleobases are linked N-glycosidically with carbon atom 1 of the ribose, adenosines, guanosines, cytidines and thymidines being formed in the individual case. In the acids, a phosphate group links the 5′-hydroxyl group of the nucleosides with the 3′-OH group of the following nucleoside in each case by means of a phosphodiester bridge with formation of single-stranded DNA or RNA. Because of the large ratio of length to diameter, DNA and RNA molecules are prone, even on mechanical stress, for example during extraction, to strand breakage. For this reason, the molecular weight of the nucleic acids can reach 10³ to 10⁹ daltons. Within the meaning of the invention, concentrated DNA and RNA solutions are employed, which are distinguished by a liquid-crystalline behavior. Preferably, deoxy- and ribonucleic acids are employed which are obtained from marine sources, for example by extraction of fish sperm, and which have a molecular weight in the region from 40,000 to 1,000,000 daltons.

The mixtures of active agents of the invention can contain the phytostenol esters (a) and the potentiating agents (b) in a ratio by weight of from 99:1 to 1:99, preferably from 90:10 to 10:90, in particular from 70:25 to 25:75 and particularly preferably from 60:40 to 40:60, where the only thing that has to be made sure is that, with the use according to the invention, an amount of the component (a) which is sufficient for lowering the cholesterol content in the blood is administered. In a special embodiment of the invention, the phytostenol esters—on their own or together with the potentiating agents—are encapsulated in a manner known per se in gelatin, the components (a) and, if appropriate, (b) being in each case employed in amounts of from 0.1 to 50, preferably from 1 to 30, in particular from 5 to 25 and particularly preferably from 10 to 15% by weight, based on the weight of the gelatin capsules. A further aspect of the invention relates to the finding that the encapsulation of the phytostenol esters in gelatin is an advantageous embodiment for oral administration of the active agents.

A further administration form of the phytostenol esters are suppositories which can be introduced rectally or vaginally and which may, as suppository base, likewise comprise gelatin, if appropriate in combination with glycerol, or else synthetic fats and/or waxes, polyethylene glycols or natural components, such as, for example, cocoa butter. In addition, it is possible to dissolve or disperse the phytostenol esters in customary foodstuffs, such as, for example: salad oils, dressings, mayonnaises, margarines, butter, deep-frying fats, cocoa products, sausage and the like.

### EXAMPLES

### Examples 1 to 5, Comparative Examples C1 to C5

Gelatin capsules (weight about 1.5 g) having a content of 5% by weight of various β-sitostenol esters and, if appropriate Vitamin E and also 0.5% by weight of radiolabeled cholesterol were prepared. To investigate the hypocholesteremic action, male rats (individual weight about 200 g) were allowed to fast overnight. The following day, a comminuted gelatin capsule was introduced into the experimental animals in each case with some salt-containing water by means of a stomach tube. After 3, 6, 12, 24 and 48 h, blood was taken from the animals and the content of radioactive cholesterol was determined. The results, which represent the mean value of the measurements of 10 experimental animals, are summarized in Table 1. The details on the decrease in the radioactivity are in each case interpreted with respect to a blind group of experimental animals, to which only gelatin capsules having a content of 20% by weight of vitamin E and an appropriate amount of radiolabeled cholesterol had been administered. The mixtures 1 to 5 are according to the invention; the mixtures C1 to C3 serve for comparison.

TABLE 1
Hypocholesteremic action (quantitative data as % by
weight based on gelatin capsule)

| Composition/activity | 1 | 2 | 3 | 4 | 5 | C1 | C2 | C3 |
|---|---|---|---|---|---|---|---|---|
| Conjuene fatty acid β-sitostenol ester* | 5 | — | — | — | — | — | — | — |
| Conj. C₁₂-C₂₄-fish fatty acid β- | — | 5 | — | — | — | — | — | — |
| sitostenol ester | | | | | | | | |
| Conjuene fatty acid β-sitostanol ester* | — | — | 5 | — | — | — | — | — |
| Conj. C₁₂-C₂₄-fish fatty acid β- | — | — | — | 5 | 5 | — | — | — |
| sitostenol ester | | | | | | | | |
| Lauric acid β-sitostanol ester | — | — | — | — | — | — | — | — |
| Oleic acid β-sitostanol ester | — | — | — | — | — | 5 | — | — |
| Linoleic acid β-sitostanol ester | — | — | — | — | — | — | 5 | — |
| Vitamin E | — | — | — | — | 5 | — | — | 5 |
| Radioactivity [%-rel] | | | | | | | | |
| after 3 h | 95 | 95 | 95 | 95 | 95 | 95 | 95 | 95 |
| after 6 h | 80 | 79 | 78 | 78 | 75 | 84 | 82 | 83 |
| after 12 h | 72 | 70 | 68 | 67 | 61 | 76 | 74 | 73 |
| after 24 h | 45 | 45 | 43 | 43 | 39 | 51 | 48 | 47 |
| after 48 h | 21 | 20 | 18 | 17 | 15 | 30 | 26 | 25 |

*fatty acid base: Selin ® CLA (Grünau/Illertissen)

What is claimed is:

1. A method of reducing serum cholesterol content in a mammal, said method comprising:
(i) providing a hypocholesteremic preparation comprising at least one phytostenol ester of a conjugated fatty acid having from about 6 to about 24 carbon atoms; and
(ii) administering the hypocholesteremic preparation to a mammal in an amount effective to reduce serum cholesterol content in the mammal.

2. The method according to claim 1, wherein the at least one phytostenol ester comprises an ester of β-sitostenol or β-sitostanol.

3. The method according to claim 1, wherein the conjugated fatty acid is selected from the group consisting of conjugated linoleic acid and conjugated fish fatty acids.

4. The method according to claim 1, wherein the conjugated fatty acid comprises conjugated linoleic acid.

5. The method according to claim 1, wherein the at least one phytostenol ester comprises an ester of conjugated linoleic acid and β-sitostenol or β-sitostanol.

6. The method according to claim 1, wherein the hypocholesteremic preparation further comprises a potentiating agent selected from the group consisting of tocopherols, chitosans, phytostenol sulfates, (deoxy)ribonucleic acids, and combinations thereof.

7. The method according to claim 1, wherein the hypocholesteremic preparation further comprises a tocopherol.

8. The method according to claim 1, wherein the hypocholesteremic preparation further comprises a chitosan selected from low-molecular weight chitosans and high-molecular weight chitosans.

9. The method according to claim 1, wherein the hypocholesteremic preparation is encapsulated in gelatin, whereby a gelatin capsule is provided, prior to administering the preparation to the mammal.

10. The method according to claim 1, wherein the at least one phytostenol ester is present in an amount of from about 0.1 to about 50% by weight, based on the total weight of the gelatin capsule.

11. A hypocholesteremic preparation comprising at least one phytostenol ester of a conjugated fatty acid having from about 6 to about 24 carbon atoms.

12. The hypocholesteremic preparation according to claim 11, wherein the at least one phytostenol ester comprises an ester of β-sitostenol or β-sitostanol.

13. The hypocholesteremic preparation according to claim 11, wherein the conjugated fatty acid is selected from the group consisting of conjugated linoleic acid and conjugated fish fatty acids.

14. The hypocholesteremic preparation according to claim 11, wherein the conjugated fatty acid comprises conjugated linoleic acid.

15. The hypocholesteremic preparation according to claim 11, wherein the at least one phytostenol ester comprises an ester of conjugated linoleic acid and β-sitostenol or β-sitostanol.

16. The hypocholesteremic preparation according to claim 11, wherein the hypocholesteremic preparation further comprises a potentiating agent selected from the group consisting of tocopherols, chitosans, phytostenol sulfates, (deoxy)ribonucleic acids, and combinations thereof.

17. The hypocholesteremic preparation according to claim 11, wherein the hypocholesteremic preparation further comprises a tocopherol.

18. The hypocholesteremic preparation according to claim 11, wherein the hypocholesteremic preparation further comprises a chitosan selected from low-molecular weight chitosans and high-molecular weight chitosans.

19. The hypocholesteremic preparation according to claim 11, wherein the hypocholesteremic preparation is encapsulated in gelatin, whereby a gelatin capsule is provided.

20. The method according to claim 19, wherein the at least one phytostenol ester is present in an amount of from about 0.1 to about 50% by weight, based on the total weight of the gelatin capsule.
